(19) United States
(12) Patent Application Publication
(10) Pub. No.: US 2006/0057095 A1
(43) Pub. Date: Mar. 16, 2006

(54) Cosmetic composition comprising at least one cationic surfactant, at least one cationic polymer, at least one fatty alcohol, and at least one diol

(76) Inventors: Pascale Lazzeri, Levallois Perret (FR); Sandrine Decoster, Saint Gratien (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/219,772

(22) Filed: Sep. 7, 2005

Related U.S. Application Data
(60) Provisional application No. 60/612,874, filed on Sep. 27, 2004.

(30) Foreign Application Priority Data
Sep. 8, 2004 (FR) 04 51989

(51) Int. Cl. A61K 8/41

(52) U.S. Cl. 424/70.28

(57) ABSTRACT

Disclosed herein is a cosmetic composition, for example, a hair composition, comprising, in a cosmetically acceptable medium, at least one cationic surfactant, at least one cationic polymer, at least one fatty alcohol, and at least one diol comprising 6 carbon atoms, wherein the total concentration of the at least one fatty alcohol is greater than or equal to 4% by weight relative to the total weight of the composition.

This application claims benefit of U.S. Provisional Application No. 60/612,874, filed Sep. 27, 2004, the contents of which are incorporated herein by reference. This application also claims benefit of priority under 35 U.S.C. § 119 to French Patent Application No. 0451989, filed Sep. 8, 2004, the contents of which are also incorporated by reference.

Disclosed herein are novel cosmetic compositions, for example, hair compositions, comprising at least one cationic surfactant, at least one cationic polymer, at least one fatty alcohol, and at least one diol. Also disclosed herein is the use of these compositions in cosmetics, for example, as hair conditioners.

Standard hair conditioners comprising a cationic surfactant and fatty alcohols are known to allow good cosmetic properties to be obtained for the hair. However, the user occasionally has the sensation that his hair is heavy and lank, and easily becomes greasy again, particularly if his hair is fine. In addition, for certain hair types, the cosmetic properties obtained may be insufficient. Furthermore, the product may be occasionally difficult to distribute.

The present inventors have discovered that compositions comprising at least one cationic surfactant, at least one non-silicone cationic polymer, at least one fatty alcohol present in an amount greater than or equal to 4% by weight relative to the total weight of the composition, and at least one diol comprising six carbon atoms, may overcome the drawbacks mentioned above.

These compositions may also allow for softening of the fiber, i.e., they may make the hair fiber less coarse and more malleable.

In addition, during application to fibers such as the hair, the compositions disclosed herein may be distributed and spread easily from the roots to the ends, and may be easier to rinse out.

Finally, the compositions disclosed herein may give the fibers, such as hair, good cosmetic properties, for example, improved disentangling and/or smoothness, such that the hair is light and/or becomes greasy again less quickly, and/or the hair may be shaped more easily and/or durably.

One embodiment of the present disclosure is thus a cosmetic composition, for example, a hair composition, comprising, in a cosmetically acceptable medium, at least one cationic surfactant chosen from quaternary ammonium salts, at least one non-silicone cationic polymer, at least one fatty alcohol, and at least one diol comprising six carbon atoms, wherein the at least one fatty alcohol is present in an amount greater than or equal to 4% by weight relative to the total weight of the composition.

Another embodiment of the present disclosure relates to a cosmetic haircare process comprising applying the compositions according to the present disclosure to the hair.

A further embodiment of this disclosure is a cosmetic treatment process comprising applying the compositions of the present disclosure to the hair and the scalp.

Yet another embodiment of the instant disclosure is a process for conditioning keratin materials such as the hair comprising applying the compositions disclosed herein to the keratin materials.

Other characteristics, aspects, subjects and advantages of the present disclosure will emerge even more clearly on reading the description and the examples that follow.

As used herein, the term “at least one” will be understood as meaning “one or more.”

As used herein, the term “cosmetically acceptable medium” means a medium that is compatible with keratin materials, such as, for example, the skin, the hair, the nails, the eyelashes, the eyebrows, the lips, and/or any other area of body or facial skin.

The cosmetically acceptable medium may comprise water, at least one cosmetically acceptable solvent, which is, in at least one embodiment, hydrophilic, or water-solvent mixtures. These solvents may be alcohols, such as linear or branched C₁-C₆ monoalcohols, for example ethanol, tert-butanol, n-butanol, isopropanol, and n-propanol.

The cosmetically acceptable medium may be present in the composition in an amount ranging from 30% to 99% by weight, for example from 60% to 90% by weight, relative to the total weight of the composition.

Cationic Surfactants

Among the quaternary ammonium salts that may be suitable for use as cationic surfactants in the compositions of this disclosure are alkylpyridinium salts, imidazoline ammonium salts, diquaternary ammonium salts, and ammonium salts comprising at least one ester function.

In at least one aspect, the cationic surfactants may be monomeric, i.e., neither the cation nor the anion is an oligomer or a polymer.

Examples of suitable quaternary ammonium salts include, but are not limited to:

quaternary ammonium salts of formula (V) below: 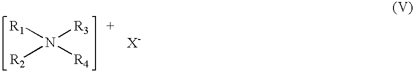
in which:

R₁, R₂, R₃, and R₄, which may be identical or different, are chosen from linear or branched aliphatic radicals comprising from 1 to 30 carbon atoms and from aromatic radicals such as aryl and alkylaryl radicals. The aliphatic radicals may comprise hetero atoms, such as oxygen, nitrogen, and sulphur, and/or halogen atoms. The aliphatic radicals may be chosen, for example, from alkyl, alkoxy, C₂-C₆ polyoxyalkylene, alkylamide, (C₁₂-C₂₂)alkylamido(C₂-C₆)alkyl, (C₁₂-C₂₂)alkylacetate, and hydroxyalkyl radicals, comprising from 1 to 30 carbon atoms; and

X⁻ is an anion chosen from halides, phosphates, acetates, lactates, (C₂-C₆) alkyl sulphates, alkylsulphonates, and alkylaryl-sulphonates;

quaternary ammonium salts of imidazoline, for instance those of formula (VI) below: 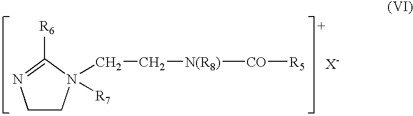
in which

R₅ is chosen from alkenyl and alkyl radicals comprising from 8 to 30 carbon atoms, for example fatty acid derivatives of tallow or of coconut,

R₆ is chosen from hydrogen, C₁-C₄ alkyl radicals, and alkenyl and alkyl radicals comprising from 8 to 30 carbon atoms,

R₇ is chosen from C₁-C₄ alkyl radicals,

R₈ is chosen from hydrogen and C₁-C₄ alkyl radicals, and

X⁻ is an anion chosen from halides, phosphates, acetates, lactates, alkyl sulphates, alkyl sulphonates, and alkylaryl sulphonates. In one embodiment of the present disclosure, R₅ and R₆ are independently chosen from mixtures of alkenyl or alkyl radicals compising from 12 to 21 carbon atoms, such as, for example, fatty acid derivatives of tallow, R₇ is methyl, and R₈ is hydrogen. Commercial products include, for example, Quaternium-27 (CTFA 1997) and Quaternium-83 (CTFA 1997), which are sold under the names “Rewoquat®” W75, W90, W75PG, and W75HPG by the company Witco;

diquaternary ammonium salts of formula (VII): 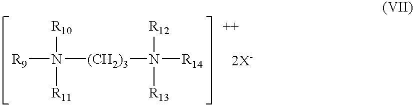
in which

R₉ is chosen from aliphatic radicals comprising from 16 to 30 carbon atoms,

R₁₀, R₁₁, R₁₂, R₁₃, and R₁₄, which may be identical or different, are chosen from hydrogen and alkyl radicals comprising from 1 to 4 carbon atoms, and

X⁻ is an anion chosen from halides, acetates, phosphates, nitrates, ethyl sulphates, and methyl sulphates. A non-limiting example of such diquaternary ammonium salts is propanetallowdiammonium dichloride.

quaternary ammonium salts comprising at least one ester function, such as those of formula (VIII) below: 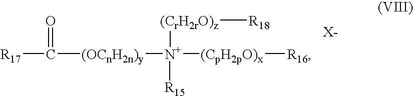
in which:

R₁₅ is chosen from C₁-C₆ alkyl radicals and C₁-C₆ hydroxyalkyl and dihydroxyalkyl radicals;

R₁₆ is chosen from 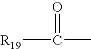
radicals, linear or branched, saturated or unsaturated C₁-C₂₂ hydrocarbon-based radicals R₂₀, and hydrogen;

R₁₇ is chosen from 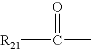
radicals; linear or branched, saturated or unsaturated C₁-C₆ hydrocarbon-based radicals R₂₂, and hydrogen;

R₁₇, R₁₉, and R₂₁, which may be identical or different, are chosen from linear or branched, saturated or unsaturated C₇-C₂, hydrocarbon-based radicals;

r, n, and p, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z ranges from 1 to 15, that when x is 0, then R₁₆ denotes R₂₀, and that when z is 0, then R₁₈ denotes R₂₂.

The alkyl radicals R₁₅ may be linear or branched. In at least one embodiment, the alkyl radicals R₁₅ are linear. In another embodiment, R₁₅ may be chosen from methyl, ethyl, hydroxyethyl, and dihydroxypropyl radicals. For example, R₁₅ may be chosen from methyl and ethyl radicals.

According to at least one embodiment of the instant disclosure, the sum x+y+z may range from 1 to 10.

In another embodiment, when R₁₆ is a hydrocarbon-based radical R₂₀, it may comprise, for example, from 12 to 22 carbon atoms, or from 1 to 3 carbon atoms.

In another embodiment, when R₁₈ is a hydrocarbon-based radical R₂₂, it may comprise from 1 to 3 carbon atoms.

According to another embodiment of the present disclosure, R₁₇, R₁₉, and R₂₁, which may be identical or different, may be chosen from linear or branched, saturated or unsaturated C₁₁-C₂₁ hydrocarbon-based radicals, for example, linear or branched, saturated or unsaturated C₁₁-C₂₁ alkyl and alkenyl radicals.

In a further embodiment of the disclosure, x and z, which may be identical or different, are equal to 0 or 1. In another embodiment, y is equal to 1. In yet another embodiment, r, n, and p, which may be identical or different, are equal to 2 or 3. In still a further embodiment, r, n, and p are equal to 2.

The anion X⁻ may be chosen from halides, for example, chloride, bromide, and iodide, and C₁-C₄ alkyl sulphates, such as methyl sulphate. Other suitable anions include, but are not limited to, methanesulphonate, phosphate, nitrate, tosylate, anions derived from organic acids, such as acetate or lactate, and any other anions that are compatible with the ammonium comprising an ester function may be used. In one embodiment, the anion X⁻ is chosen from chloride and methyl sulphate.

Other suitable quaternary ammonium salts include those of formula (VIII) in which:

R₁₅ is chosen from methyl and ethyl radicals;

x and y are equal to 1;

z is equal to 0 or 1;

r, n, and p are equal to 2;

R₁₆ is chosen from 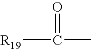
radicals, methyl radicals, ethyl radicals, C₁₄-C₂₂ hydrocarbon-based radicals, and hydrogen;

R₁₈ is chosen from 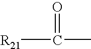
radicals and hydrogen;

R₁₇, R₁₉, and R₂₁, which may be identical or different, are chosen from linear or branched, saturated or unsaturated C₁₃-C₁₇ hydrocarbon-based radicals, for example, linear or branched, saturated or unsaturated C₁₃-C₁₇ alkyl and alkenyl radicals. In one embodiment, the C₁₃-C₁₇ hydrocarbon-based radicals are linear.

Examples of compounds of formula (VIII) include salts (for example, chlorides and methyl sulphates) of diacyloxyethyl-dimethylammonium, diacyloxyethyl-hydroxyethyl-methylammonium, monoacyloxyethyl-dihydroxyethyl-methylammonium, triacyloxyethyl-methylammonium, monoacyloxyethyl-hydroxyethyl-dimethylammonium, and mixtures thereof. In one embodiment, the acyl radicals may comprise 14 to 18 carbon atoms and may be derived from plant oils, for instance, palm oil and sunflower oil. When the compound comprises several acyl radicals, these radicals may be identical or different.

These products may be obtained, for example, by direct esterification of optionally oxyalkylenated triethanolamine, triisopropanolamine, alkyldiethanolamine, or alkyldiisopropanolamine onto fatty acids or onto mixtures of fatty acids of plant or animal origin, or by transesterification of the methyl esters thereof. This esterification may be followed by a quaternization using at least one alkylating agent chosen from alkyl halides (for example, methyl halides and ethyl halides), dialkyl sulphates (for example, dimethyl sulphate and diethyl sulphate), methyl methanesulphonate, methyl para-toluenesulphonate, glycol chlorohydrin, and glycerol chlorohydrin.

Such compounds are sold, for example, under the names Dehyquart® by the company Cognis, Stepanquat® by the company Stepan, Noxamium® by the company Ceca, and Rewoquat® WE 18 by the company Rewo-Goldschmidt.

The composition according to the present disclosure may comprise a mixture of quaternary ammonium mono-, di-, and triester salts, with a weight majority of diester salts.

Examples of mixtures of ammonium salts that may be used include, but are not limited to, mixtures comprising 15% to 30% by weight of acyloxyethyl-dihydroxyethyl-methylammonium methyl sulphate, 45% to 60% of diacyloxyethyl-hydroxyethyl-methylammonium methyl sulphate, and 15% to 30% of triacyloxyethyl-methylammonium methyl sulphate, wherein the acyl radicals may comprise from 14 to 18 carbon atoms and may be derived from optionally partially hydrogenated palm oil.

It is also possible to use ammonium salts comprising at least one ester function, such as those described in U.S. Pat. Nos. 4,874,554, and 4,137,180.

In at least one embodiment of the present disclosure, the quaternary ammonium salts corresponding to formula (V) are used in the compositions described herein. Mention may be made of salts (for example, methosulphates) of dipalmitoyl-ethylhydroxymethylammonium; salts (for example, chlorides) of tetraalkylammonium, for instance, salts (such as chlorides) of dialkyldimethylammonium and alkyl-trimethylammonium in which the alkyl radical comprises from 12 to 22 carbon atoms, such as behenyltrimethylammonium, distearyidimethylammonium, cetyltrimethylammonium and benzyldimethylstearylammonium salts (for example, chlorides). In addition, mention may be made of salts (for example, chlorides) of palmitylamidopropyltrimethylammonium and salts (for example, chlorides) of stearamidopropyldimethyl(myristyl acetate)ammonium, such as the stearamidopropyldimethyl(myristyl acetate)ammonium chloride sold under the name Ceraphyl® 70 by the company Van Dyk.

In at least one embodiment, the cationic surfactants used in the presently disclosed compositions are quaternary ammonium salts chosen from behenyltrimethylammonium chloride, cetyltrimethylammonium chloride, dipalmitoyl-ethylhydroxyethylmethylammonium chloride, cetyltrimethylammonium chloride, Quaternium-83, behenylamidopropyl-2,3-dihydroxypropyldimethylammonium chloride, and palmitylamidopropyltrimethylammonium chloride.

The at least one cationic surfactant may be present in the composition in an amount ranging from 0.01% to 10% by weight, for example, from 0.1% to 5% by weight, or from 0.2% to 4% by weight, relative to the total weight of the composition.

Fatty Alcohols

The at least one fatty alcohol according to the present disclosure may be linear or branched, saturated or unsaturated, and may comprise from 8 to 40 carbon atoms. In at least one embodiment, the at least one fatty alcohol may also be oxyalkylenated or glycerolated.

The fatty alcohol may have the structure R—OH, in which R is chosen from saturated or unsaturated, linear or branched radicals comprising from 8 to 40 carbon atoms, for example, from 8 to 30 carbon atoms. In one embodiment, R is chosen from C₈-C₄₀, for example, C₁₂-C₂₄, alkyl and alkenyl groups. R may be substituted with at least one hydroxyl group, for example, with one or two hydroxyl groups. In another embodiment, R is not substituted with a hydroxyl group.

Examples of suitable fatty alcohols include, but are not limited to cetyl alcohol, stearyl alcohol, behenyl alcohol, isocetyl alcohol, isostearyl alcohol, isobehenyl alcohol, oleyl alcohol, and mixtures thereof. In one embodiment, the alcohol may be chosen from cetyl alcohol, stearyl alcohol, behenyl alcohol, and mixtures thereof.

As used herein, the term “oxyalkylenated fatty alcohol” means any pure fatty alcohol having the following structure: 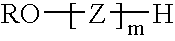
in which:

R is chosen from saturated or unsaturated, linear or branched radicals comprising from 8 to 40 carbon atoms, for example, from 8 to 30 carbon atoms,

Z is an oxyethylene radical of formula (i), and/or an oxypropylene radical chosen from oxypropylene radicals of formulas (ii)₁ and (ii)₂:

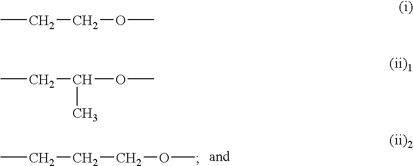

m is the number of ethylene oxide and/or propylene oxide groups, and may range from 1 to 250, for example, from 2 to 100.

As used herein, the term “glycerolated fatty alcohol” means any pure fatty alcohol having the following structure: 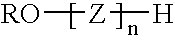
in which:

R is chosen from saturated or unsaturated, linear or branched radicals comprising from 8 to 40 carbon atoms, for example, from 8 to 30 carbon atoms,

Z is a glycerolated radical of formula (iii):

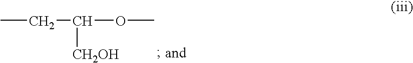

n is the number of glycerol groups, and may range from 1 to 30, for example, from 1 to 10.

In at least one embodiment of the present disclosure, the oxyalkylenated fatty alcohols according to the present disclosure may be saturated or unsaturated, linear or branched fatty alcohols comprising from 10 to 20 carbon atoms and from 2 to 40 ethylene oxide groups.

Non-limiting examples of oxyalkylenated fatty alcohols include the following marketed products:

MERGITAL LM2 (Cognis) [lauryl alcohol 2 EO];

IFRALAN L12 (Ifrachem) and REWOPAL 12 (Goldschmidt) [lauryl alcohol 12 EO];

EMPILAN KA 2.5/90FL (Albright & Wilson) and MERGITAL BL309 (Cognis) [decyl alcohol 3 EO];

EMPILAN KA 5/90 FL (Albright & Wilson) and MERGITAL BL589 (Cognis) [decyl alcohol 5 EO];

BRIJ 58 (Uniquema) and SIMULSOL 58 (SEPPIC) [cetyl alcohol 20 EO];

EMULGIN 05 (Cognis) [oleocetyl alcohol 5 EO];

MERGITAL OC30 (Cognis) [oleocetyl alcohol 30 EO];

BRIJ 72 (Uniquema) [stearyl alcohol 2 EO];

BRIJ 76 (Uniquema) [stearyl alcohol 10 EO];

BRIJ 78P (Uniquema) [stearyl alcohol 20 EO];

BRIJ 700 (Uniquema) [stearyl alcohol 100 EO];

EMULGIN B1 (Cognis) [cetylstearyl alcohol 12 EO];

EMULGIN L (Cognis) [cetyl alcohol 9 EO and 2 PO]; and

WITCONOL APM (Goldschmidt) [myristyl alcohol 3 PO].

Examples of glycerolated fatty alcohols include, but are not limited to, lauryl alcohol containing 4 mol of glycerol (INPCI name: Polyglyceryl-4 Lauryl Ether), oleyl alcohol containing 4 mol of glycerol (INPCI name: Polyglyceryl-4 Oleyl Ether), oleyl alcohol containing 2 mol of glycerol (INPCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The at least one fatty alcohol may also be a mixture of fatty alcohols, which means that several fatty alcohol species may coexist in a commercial product, in the form of a mixture.

In at least one embodiment, the fatty alcohol may be solid or pasty at a temperature of 20° C. As used herein, the expression “fatty alcohol that is solid or pasty at 20° C.” means an alcohol that has a viscosity, measured with a rheometer at a shear rate of 1 s⁻¹, of greater than or equal to 1 Pa·s.

The at least one fatty alcohol of the present disclosure may be non-oxyalkylenated and/or non-glycerolated.

The at least one fatty alcohol may be present in the composition in an amount ranging from 4% to 15%, for example, from 4.5% to 10%, or from 4.5% to 8% by weight relative to the total weight of the composition.

Diols

As used herein, the term “diol comprising 6 carbon atoms” means any hydrocarbon-based compound comprising two hydroxyl functions and six carbon atoms.

The at least one diol may be linear or branched and may comprise ether functions. The at least one diol may be chosen from, for example, 1,6-hexanediol, dipropylene glycol, and mixtures thereof.

The at least one diol may be present in the composition in an amount ranging from 0.1% to 10%, for example, from 0.25% to 5%, or from 0.5% to 3% by weight relative to the total weight of the composition.

Cationic Polymer

According to the present disclosure, the compositions described herein may also comprise at least one non-silicone cationic polymer. As used herein, the term “non-silicone” means that the cationic polymer does not comprise any (Si—O) units.

The cationic polymers that may be used in accordance with the present disclosure may be chosen from those already known per se as improving the cosmetic properties of the hair, for example, those described in European Patent Application No. 0 337 354 A and French Patent Application Nos. 2 270 846, 2 383 660, 2 598 611, 2 470 596, and 2 519 863.

As used herein, the term “cationic polymer” denotes any polymer comprising at least one group chosen from cationic groups and groups that may be ionized into cationic groups.

The cationic polymers may be chosen from those comprising units comprising primary, secondary, tertiary, and/or quaternary amine groups that either may form part of the main polymer chain or may be borne by a side substituent directly attached thereto.

The cationic polymers may have a number-average molar mass ranging from 500 to 5×10⁶, for example, from 10³ to 3×10⁶.

Non-limiting examples of suitable cationic polymers include polymers known in the art, such as polyamine, polyamino amide, and polyquaternary ammonium polymers.

Examples of polyaminoamide and polyquaternary ammonium polymers that may be used in accordance with the present disclosure include, but are not limited to, those described in French Patent Nos. 2 505 348 and 2 542 997. For example, these polymers may be chosen from:

(1) homopolymers or copolymers derived from acrylic or methacrylic esters and amides, comprising at least one of the units of the following formulae: 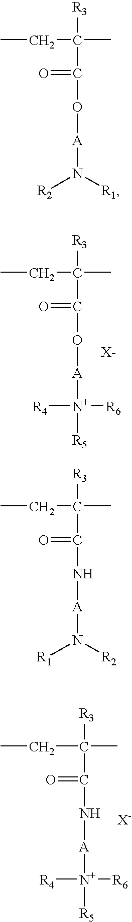
in which:

R₃, which may be identical or different, are chosen from hydrogen and CH₃ radicals;

A, which may be identical or different, are chosen from linear or branched alkyl groups comprising from 1 to 6 carbon atoms, for example, from 2 or 3 carbon atoms, and hydroxyalkyl groups comprising from 1 to 4 carbon atoms;

R₄, R₅, and R₆, which may be identical or different, are chosen from alkyl groups comprising from 1 to 18 carbon atoms, for example, 1 to 6 carbon atoms, and benzyl radicals;

R₁ and R₂, which may be identical or different, are chosen from hydrogen and alkyl groups comprising from 1 to 6 carbon atoms, for example, methyl and ethyl groups;

X⁻ is chosen from anions derived from mineral or organic acids, for example, methosulphate anions, and halides such as chloride and bromide.

The copolymers of family (1) may also comprise at least one unit derived from comonomers chosen from acrylamides; methacrylamides; diacetone acrylamides; acrylamides and methacrylamides substituted on the nitrogen with lower (C₁-C₄) alkyls; acrylic or methacrylic acids and esters thereof; vinyllactams, such as vinylpyrrolidone and vinylcaprolactam; and vinyl esters.

Thus, the copolymers of family (1), may include, but are not limited to:

copolymers of acrylamide and of dimethylaminoethyl methacrylate quaternized with dimethyl sulphate or with a dimethyl halide, such as the product sold under the name HERCOFLOC by the company Hercules,

copolymers of acrylamide and of methacryloyloxyethyl-trimethylammonium chloride described, for example, in European Patent Application No. 080 976 A and sold under the name BINA QUAT P 100 by the company Ciba-Geigy,

copolymers of acrylamide and of methacryloyloxyethyl-trimethylammonium methosulphate, such as the product sold under the name RETEN by the company Hercules,

quaternized or non-quaternized vinylpyrrolidone/dialkylaminoalkyl acrylate or methacrylate copolymers, such as those described in French Patent Nos. 2 077 143 and 2 393 573,

dimethylaminoethyl methacrylate/vinylcaprolactam/vinylpyrrolidone terpolymers,

vinylpyrrolidone/methacrylamidopropyldimethylamine copolymers, and

quaternized vinylpyrrolidone/dimethylaminopropylmethacrylamide copolymers.

(2) cationic polysaccharides, for example, cationic celluloses and cationic galactomannan gums. Examples of suitable cationic polysaccharides include, but are not limited to, cellulose ethers comprising quaternary ammonium groups, cationic cellulose copolymers or celluloses grafted with a water-soluble quaternary ammonium monomer, and cationic galactomannan gums.

Non-limiting examples of cellulose ethers comprising quaternary ammonium groups are described, for example, in French Patent No. 1 492 597. These polymers are also defined in the CTFA dictionary as hydroxyethylcellulose quaternary ammoniums that have reacted with an epoxide substituted with a trimethylammonium group.

Suitable cationic cellulose copolymers or cellulose derivatives grafted with a water-soluble quaternary ammonium monomer include, but are not limited to, those described in U.S. Pat. No. 4,131,576, such as hydroxyalkylcelluloses, for instance, hydroxymethyl-, hydroxyethyl-, and hydroxypropylcelluloses grafted with a salt chosen from methacryloylethyltrimethylammonium, methacrylamidopropyltrimethylammonium, and dimethyldiallylammonium salts.

Non-limiting examples of cationic galactomannan gums include those described in U.S. Pat. Nos. 3,589,578 and 4,031,307, for example, guar gums comprising trialkylammonium cationic groups such as guar gums modified with a salt (for example, a chloride) of 2,3-epoxypropyltrimethylammonium.

(3) polymers comprising piperazinyl units and divalent alkylene or hydroxyalkylene radicals comprising straight or branched chains, optionally interrupted with at least one entity chosen from oxygen, sulphur, nitrogen, aromatic rings, and heterocyclic rings, and also the oxidation and/or quaternization products of these polymers. Examples of such polymers may be found, for instance, in French Patent Nos. 2 162 025 and 2 280 361;

(4) water-soluble polyamino amides prepared, for example, by polycondensation of an acidic compound with a polyamine; these polyamino amides may be crosslinked with at least one crosslinking agent chosen from epihalohydrins; diepoxides; dianhydrides; unsaturated dianhydrides; bis-unsaturated derivatives; bis-halohydrins; bis-azetidiniums; bis-haloacyidiamines; bis-alkyl halides; and oligomers resulting from the reaction of a difunctional compound which is reactive with at least one entity chosen from bis-halohydrins, bis-azetidiniums, bis-haloacyldiamines, bis-alkyl halides, epihalohydrins, diepoxides, and bis-unsaturated derivatives; the crosslinking agent being used in an amount ranging from 0.025 to 0.35 mol per amine group of the polyamino amide. These polyamino amides may be alkylated or, if they comprise at least one tertiary amine function, they may be quaternized. Non-limiting examples of such polymers may be found, for example, in French Patent Nos. 2 252 840 and 2 368 508;

(5) polyaminoamide derivatives resulting from the condensation of polyalkylene polyamines with polycarboxylic acids followed by alkylation with difunctional agents. Examples of such polyaminoamide derivatives include, but are not limited to, adipic acid/dialkylaminohydroxyalkyldialkylenetriamine polymers in which the alkyl radical comprises from 1 to 4 carbon atoms, for example, methyl, ethyl, and propyl. Such polymers are described, for example, in French Patent No. 1 583 363.

Further examples of these polyaminoamides include the adipic acid/dimethylaminohydroxypropyl/diethylenetriamine polymers sold under the name “CARTARETINE F, F4 or F8” by the company Sandoz.

(6) polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms, wherein the molar ratio between the polyalkylene polyamine and the dicarboxylic acid may range from 0.8:1 to 1.4:1; the polyamino amide resulting therefrom being reacted with epichlorohydrin in a molar ratio of epichlorohydrin relative to the secondary amine group of the polyamino amide ranging from 0.5:1 to 1.8:1. Examples of such polymers may be found in U.S. Pat. Nos. 3,227,615 and 2,961,347.

Other non-limiting examples of polymers of this type include those sold under the name “HERCOSETT 57” by the company Hercules Inc. in the case of the adipic acid/epoxypropyl/diethylenetriamine copolymer.

(7) cyclopolymers of alkyldiallylamine or of dialkyldiallylammonium, such as the homopolymers or copolymers comprising, as main constituent of the chain, at least one unit chosen from units of formula (I) and units of formula (I′): 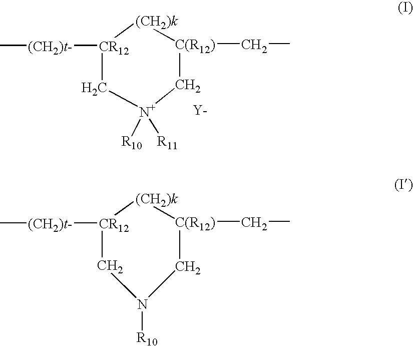
in which:

k and t are equal to 0 or 1, wherein the sum k+t is equal to 1;

R₁₂ is chosen from hydrogen and from methyl radicals;

R₁₀ and R₁₁, independently of each other, are chosen from alkyl groups comprising from 1 to 6 carbon atoms; hydroxyalkyl groups, for example, hydroxyalkyl groups in which the alkyl group comprises from 1 to 5 carbon atoms; and lower C₁-C₄ amidoalkyl groups, or alternatively R₁₀ and R₁₁ may form, together with the nitrogen atom to which they are attached, heterocyclic groups, such as piperidyl and morpholinyl; and

Y⁻ is an anion chosen from bromide, chloride, acetate, borate, citrate, tartrate, bisulphate, bisulphite, sulphate, and phosphate. Non-limiting examples of these polymers may be found in French Patent No. 2 080 759 and its Certificate of Addition 2 190 406.

In at least one embodiment of the present disclosure, R₁₀ and R₁₁, independently of each other, may be chosen from alkyl groups comprising from 1 to 4 carbon atoms.

Further examples of the polymers defined above include, but are not limited to, the dimethyldiallylammonium chloride homopolymer sold under the name “MERQUAT 100” by the company Nalco (and its homologues of low weight-average molecular mass), copolymers of diallyldimethylammonium chloride, and copolymers of diallyidimethylammonium and acrylamide.

(8) diquaternary ammonium polymers comprising at least one repeating unit of formula (II): 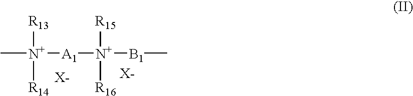
in which:

R₁₃, R₁₄, R₁₅, and R₁₆, which may be identical or different, are chosen from aliphatic, alicyclic, and arylaliphatic radicals comprising from 1 to 20 carbon atoms and lower hydroxyalkylaliphatic radicals, or alternatively R₁₃, R₁₄, R₁₅, and R₁₆, together or separately, may form, together with the nitrogen atoms to which they are attached, heterocycles optionally comprising a second hetero atom other than nitrogen, or alternatively R₁₃, R₁₄, R₁₅, and R₁₆ may form a group chosen from linear or branched C₁-C₆ alkyl radicals substituted with an entity chosen from nitrile groups, ester groups, acyl groups, amide groups; —CO—O—R₁₇-D groups, and —CO—NH—R₁₇-D groups, wherein R₁₇ is an alkylene and D is a quaternary ammonium group;

A₁ and B₁, which may be identical or different, are chosen from polymethylene groups comprising from 2 to 20 carbon atoms, which groups may be linear or branched, saturated or unsaturated, and which may comprise, linked to or intercalated in the main chain, at least one entity chosen from aromatic rings, oxygen, sulphur, sulphoxide groups, sulphone groups, disulphide groups, amino groups, alkylamino groups, hydroxyl groups, quaternary ammonium groups, ureido groups, amide groups, and ester groups; and

X⁻ is an anion derived from a mineral or organic acid;

A₁, R₁₃, and R₁₅ may form, together with the two nitrogen atoms to which they are attached, a piperazine ring;

if A₁ is chosen from linear or branched, saturated or unsaturated alkylene and hydroxyalkylene radicals, B₁ may be chosen from (CH₂)ₙ—CO-D-OC—(CH₂)ₙ— groups

in which D is chosen from:

a) glycol residues of formula: —O-Z-O—, wherein Z is chosen from linear or branched hydrocarbon-based radicals and groups corresponding to one of the following formulae:
—(CH₂—CH₂—O)ₓ—CH₂—CH₂—
[CH₂—CH(CH₃)—O]_{y}—CH₂—CH(CH₃)—

wherein x and y, which may be identical or different, are chosen from integers ranging from 1 to 4, representing a defined and unique degree of polymerization, and any number ranging from 1 to 4, representing an average degree of polymerization;

b) bis-secondary diamine residues, such as piperazine derivatives;

c) bis-primary diamine residues of formula: —NH—Y—NH—, wherein Y is chosen from linear or branched hydrocarbon-based radicals and the divalent radical
—CH₂—CH₂—S—S—CH₂—CH₂—; and

d) ureylene groups of formula: —NH—CO—NH—; and

n is an integer ranging from 1 to 8, for example, from 2 to 4.

In at least one embodiment of the present disclosure, X⁻ may be an anion such as chloride or bromide.

Polymers of this type may generally have a number-average molecular mass ranging from 1,000 to 100,000. Suitable examples of these polymers include, but are not limited to, those described in French Patent Nos. 2 320 330, 2 270 846, 2 316 271, 2 336 434, and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

Other suitable diquaternary ammonium polymers include, but are not limited to, polymers that comprise repeating units corresponding to formula (a): 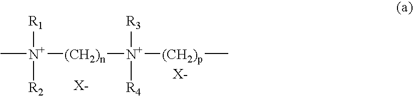
in which

R₁, R₂, R₃, and R₄, which may be identical or different, are chosen from alkyl and hydroxyalkyl radicals comprising from 1 to 4 carbon atoms,

n and p are integers ranging from 2 to 20, and

X⁻ is an anion derived from a mineral or organic acid.

In one embodiment of the present disclosure, the polymer may comprise the repeating unit of formula (a), in which R₁, R₂, R₃, and R₄ are methyl radicals, n=3, p=6, and X=Cl, which is known as Hexadimethrine chloride according to the INCI (CTFA) nomenclature.

(9) polyquaternary ammonium polymers comprising at least one unit of formula (III): 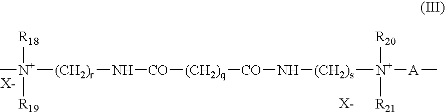
in which:

R₁₈, R₁₉, R₂₀, and R₂₁, which may be identical or different, are chosen from hydrogen, methyl radicals, ethyl radicals, propyl radicals, β-hydroxyethyl radicals, β-hydroxypropyl radicals, and —CH₂CH₂(OCH₂CH₂)ₚOH radicals,

wherein p is an integer ranging from 0 to 6, with the proviso that R₁₈, R₁₉, R₂₀, and R₂₁ are not simultaneously hydrogen,

r and s, which may be identical or different, are integers ranging from 1 to 6,

q is an integer ranging from 0 to 34,

X⁻ is an anion, such as a halide, and

A is chosen from dihalide radicals and —CH₂—CH₂—O—CH₂—CH₂—.

Examples of such compounds include, but are not limited to, those described in European Patent Application No. 0 122 324 A. Non-limiting examples of commercial products include “Mirapol® A 15,” “Mirapol® AD1,” “Mirapol® AZ1,” and “Mirapol® 175,” sold by the company Miranol.

(10) quaternary polymers of vinylpyrrolidone and vinylimidazole, for instance, the products sold under the names Luviquat® FC 905, FC-550, and FC 370 by the company BASF;

(11) optionally crosslinked methacryloyloxy(C₁-C₄)alkyltri(C₁-C₄)alkyl-ammonium salt polymers, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, for example, methylenebisacrylamide.

Other cationic polymers that may be used in in accordance with the present disclosure include cationic proteins; cationic protein hydrolysates; polyalkyleneimines, for example, polyethyleneimines; polymers comprising vinylpyridine or vinylpyridinium units; condensates of polyamines and of epichlorohydrin; quaternary polyureylenes; chitin derivatives, for example, chitosans; and salts thereof. The salts that may be used include, for example, chitosan acetate, lactate, glutamate, gluconate, and pyrrolidonecarboxylate.

The cationic polymer may be chosen from chitosans with a degree of deacetylation of 90% by weight and chitosan pyrrolidonecarboxylate, sold under the name Kytamer® PC by the company Amerchol.

In at least one aspect of the present disclosure, the at least one cationic cyclopolymer may be chosen from dimethyldiallylammonium chloride homopolymers and copolymers sold under the names “Merquat 100,” “Merquat 550,” and “Merquat S” by the company Nalco; quaternary polymers of vinylpyrrolidone and vinylimidazole; crosslinked homopolymers and copolymers of methacryloyloxy(C₁-C₄)alkyltri(C₁-C₄)alkylammonium salts; chitosan pyrrolidonecarboxylate; and mixtures thereof.

According to one embodiment of the disclosure, the at least one cationic polymer may be present in the composition in an amount ranging from 0.001% to 20% by weight, for example, from 0.01% to 10% by weight, or from 0.02% to 5% by weight relative to the total weight of the final composition.

The ratio of the weight concentrations of cationic surfactant/diol may be greater than 1, and may range, for example, from 1.5 to 10, or from 2 to 5.

The ratio of the weight concentrations of fatty alcohol/cationic surfactant may be greater than or equal to 1, and may range, for example, from 1 to 10, or from 1 to 5.

The ratio of the weight concentrations of cationic surfactant/silicone may range from 0.85 to 10, for example, from 0.85 to 5.

The ratio of the weight concentrations of fatty alcohol/silicone may range from 2 to 15, for example, from 2 to 5.

The composition according to the present disclosure may also comprise at least one additive chosen from fragrances, UV-screening agents, nonionic surfactants, anionic surfactants, amphoteric surfactants, zwitterionic surfactants, preserving agents, proteins, vitamins, provitamins, nonionic polymers, anionic polymers, cationic polymers, amphoteric polymers, zwitterionic polymers, mineral oils, plant oils, synthetic oils, silicones, plant waxes, ceramides, and any other additive conventionally used in cosmetic compositions, such as antidandruff agents, agents for preventing hair loss, dyes, pigments, reducing agents, and polyols other than those disclosed above.

The at least one additive may be present in the composition in an amount ranging from 0.001% to 20% by weight relative to the total weight of the composition. The precise amount of each additive will depend on its nature and the selected haircare application, and may be readily determined by a person skilled in the art.

The compositions according to the present disclosure may also comprise at least one silicone, for example, an amino silicone. The silicone may be chosen, for example, from amodimethicones, trimethylsilyl amodimethicones, and silicones comprising quaternary ammonium groups.

In at least one embodiment, the silicone is present in the composition in an amount ranging from 0.01% to 6%, for example, from 0.1% to 3%, or from 0.4% to 2% by weight relative to the total weight of the composition.

A person skilled in the art will take care to select the optional additives and the amount thereof such that they do not harm the properties of the compositions of the present disclosure.

The compositions according to the present disclosure may be in a form chosen from fluids, thickened liquids, gels, creams, simple emulsions, and multiple emulsions. The compositions for the cosmetic treatment of keratin materials in accordance with the present disclosure may also be in the form of a foam or mousse and may be used in rinse-out or leave-in applications. In this case, they may be packaged in an aerosol device.

The compositions of the present disclosure may be used, for example, in shampoos, hair conditioners, dyeing products, bleaching products, permanent-waving products, styling products, rinse-out care products, deep-care masks, shower gels, and scalp treatment lotions and creams, or alternatively may be deposited on wipes.

According to one embodiment of the present disclosure, the composition may be a non-coloring composition. As used herein, the term “non-coloring composition” means a composition containing essentially no oxidation dye, i.e., less than 0.5% by weight relative to the total weight of the composition, for example, less than 0.1% by weight, or a composition containing no oxidation dye.

Disclosed herein is also a method for the cosmetic treatment of the hair and/or the scalp comprising applying the compositions according to the present disclosure to the hair and/or the scalp.

Also disclosed herein is a method for conditioning keratin materials such as the hair comprising applying the compositions according to the present disclosure to the keratin materials.

Further disclosed herein is a cosmetic hair treatment method comprising applying the cosmetic composition according to the present disclosure to wet or dry hair, optionally followed by rinsing. In one embodiment, the composition may be applied after shampooing.

In another embodiment of the disclosure, the compositions according to the disclosure are used as hair conditioners for treating the hair and/or the scalp. In this case, the compositions may be applied to wet or dry hair, in amounts that are effective to treat the hair, this application optionally being followed by rinsing.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. The amounts are expressed as active material, unless otherwise indicated.

### EXAMPLE 1

A hair conditioning composition A according to the disclosure and a comparative composition B were prepared by combining the following components:

| | Composition A | | Composition B | |
|---|---|---|---|---|
| Cetylstearyl alcohol | 7 | g | 7 | g |
| Behenyltrimethylammonium chloride | 3 | g AM | 3 | g AM |
| 1,6-Hexanediol | 1 | g | — | |
| Isopropanol | — | | 1 | g |
| Mixture of myristyl/cetyl/stearyl | 2.5 | g | 2.5 | g |
| myristate/palmitate/stearate | | | | |
| Isopropyl myristate | 1.05 | g | 1.05 | g |
| Polydimethylsiloxane containing | 0.1 | g AM | 0.1 | g AM |
| aminopropyl groups as a mixture | | | | |
| with a polydimethylsiloxane | | | | |
| (KF8020 from Shin-Etsu) | | | | |
| Ethyltrimethylammonium methacrylate | 0.15 | g AM | 0.15 | g AM |
| chloride homopolymer | | | | |
| (Salcare SC 95 from Ciba) | | | | |
| Fragrance, preserving agents | qs | | Qs | |
| pH agent qs | pH 4 ± 0.5 | | pH 4 ± 0.5 | |
| Water | Qs 100 | g | qs 100 | g |

Compositions A and B were applied to the hair, followed by rinsing. Composition A was easier to rinse out than composition B.

It was observed that the hair treated with composition A was softer, livelier, and smoother than the hair treated with composition B.

### EXAMPLE 2

A hair conditioning composition A according to the disclosure and a comparative composition B were prepared by combining the following components:

| | Composition A | | Composition B | |
|---|---|---|---|---|
| Cetylstearyl alcohol | 5 | g | 5 | g |
| Behenyltrimethylammonium chloride | 3 | g AM | 3 | g AM |
| 1,6-Hexanediol | 1 | g | — | |
| Isopropanol | — | | 1 | g |
| Mixture of myristyl/cetyl/stearyl | 1 | g | 1 | g |
| myristate/palmitate/stearate | | | | |
| Isopropyl myristate | 1.05 | g | 1.05 | g |
| Polydimethylsiloxane containing | 0.5 | g AM | 0.5 | g AM |
| aminopropyl groups as a mixture | | | | |
| with a polydimethylsiloxane | | | | |
| (KF8020 from Shin-Etsu) | | | | |
| Ethyltrimethylammonium methacrylate | 0.3 | g AM | 0.3 | g AM |
| chloride homopolymer | | | | |
| (Salcare SC 95 from Ciba) | | | | |
| Fragrance, preserving agents | Qs | | Qs | |
| pH agent qs | pH 4 ± 0.5 | | pH 4 ± 0.5 | |
| Water | qs 100 | g | Qs 100 | g |

Compositions A and B were applied to the hair, followed by rinsing. Composition A was easier to rinse out than composition B.

It was observed that the hair treated with composition A was softer, livelier, and smoother than the hair treated with composition B.

### EXAMPLE 3

A hair conditioning composition A according to the disclosure was prepared by combining the following components:

| | Composition A | |
|---|---|---|
| Cetylstearyl alcohol | 7 | g |
| Behenyltrimethylammonium chloride | 3 | g AM |
| 1,6-Hexanediol | 1 | g |
| Dipropylene glycol | 1 | g |
| Mixture of myristyl/cetyl/stearyl | 2.5 | g |
| myristate/palmitate/stearate | | |
| Isopropyl myristate | 1.05 | g |
| Polydimethylsiloxane containing | 0.5 | g AM |
| aminopropyl groups as a | | |
| mixture with a polydimethylsiloxane | | |
| (KF8020 from Shin-Etsu) | | |
| Ethyltrimethylammonium methacrylate | 0.3 | g AM |
| chloride homopolymer | | |
| (Salcare SC 95 from Ciba) | | |
| Fragrance, preserving agents | qs | |
| pH agent qs | pH 4 ± 0.5 | |
| Water | qs 100 | g |

The composition spread easily onto the hair. It was observed that the hair treated with composition A was supple, lively, and smooth.

### EXAMPLE 4

A hair conditioning composition A according to the disclsosure and a comparative composition B were prepared by combining the following:

| | Composition A | | Composition B | |
|---|---|---|---|---|
| Cetyl alcohol | 4.5 | g | 4.5 | g |
| Cetyltrimethylammonium chloride | 3 | g AM | 3 | g AM |
| 1,6-Hexanediol | 1 | g AM | — | |
| Isopropanol | — | | 1 | g AM |
| Mixture of myristyl/cetyl/stearyl | 2.5 | g | 2.5 | g |
| myristate/palmitate/stearate | | | | |
| Polydimethylsiloxane containing | 0.5 | g AM | 0.5 | g AM |
| aminoethyliminobutyl | | | | |
| groups as an aqueous 58% | | | | |
| emulsion (DC2-8299 | | | | |
| from Dow Corning) | | | | |
| Glyceryl stearate | 1 | g | 1 | g |
| Fragrance, preserving agents | qs | | Qs | |
| pH agent qs | pH 4 ± 0.5 | | pH 4 ± 0.5 | |
| Water | qs 100 | g | qs 100 | g |

What is claimed is:

1. A cosmetic composition comprising, in a cosmetically acceptable medium,
at least one cationic surfactant chosen from quaternary ammonium salts,
at least one fatty alcohol,
at least one non-silicone cationic polymer, and
at least one diol comprising six carbon atoms,
wherein the at least one fatty alcohol is present in an amount greater than or equal to 4% by weight relative to the total weight of the composition.

2. The composition of claim 1, wherein the quaternary ammonium salts are chosen from:
quaternary ammonium salts of formula (V): 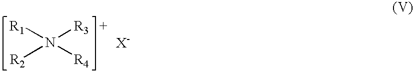
in which:
R₁, R₂, R₃, and R₄, which may be identical or different, are chosen from linear or branched aliphatic radicals comprising from 1 to 30 carbon atoms, and from aromatic radicals;
X⁻ is an anion chosen from halides, phosphates, acetates, lactates, (C₂-C₆)alkyl sulphates, alkyl sulphonates, and alkylaryl sulphonates;
quaternary ammonium salts of imidazoline of formula (VI): 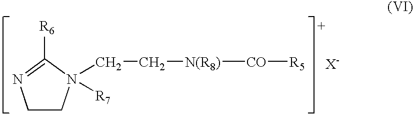
in which:
R₅ is chosen from alkenyl and alkyl radicals comprising from 8 to 30 carbon atoms,
R₆ is chosen from hydrogen, C₁-C₄ alkyl radicals, alkenyl radicals comprising from 8 to 30 carbon atoms, and alkyl radicals comprising from 8 to 30 carbon atoms,
R₇ is chosen from C₁-C₄ alkyl radicals,
R₈ is chosen from hydrogen and C₁-C₄ alkyl radicals, and
X⁻ is an anion chosen from halides, phosphates, acetates, lactates, alkyl sulphates, alkyl sulphonates, and alkylaryl sulphonates;
diquaternary ammonium salts of formula (VII): 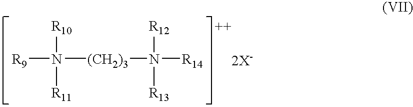
in which:
R₉ is chosen from aliphatic radicals comprising from 16 to 30 carbon atoms,
R₁₀, R₁₁, R₁₂, R₁₃, and R₁₄, which may be identical or different, are chosen from hydrogen and alkyl radicals comprising from 1 to 4 carbon atoms, and
X⁻ is an anion chosen from halides, acetates, phosphates, nitrates, ethyl sulphates, and methyl sulphates;
quaternary ammonium salts comprising at least one ester function, of formula (VIII): 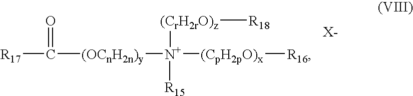
in which:
R₁₅ is chosen from C₁-C₆ alkyl radicals, C₁-C₆ hydroxyalkyl radicals, and C₁-C₆ dihydroxyalkyl radicals; R₁₆ is chosen from 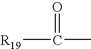
radicals, linear or branched, saturated or unsaturated C₁-C₂₂ hydrocarbon-based radicals R₂₀, and hydrogen,
R₁₇ is chosen from 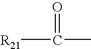
radicals, linear or branched, saturated or unsaturated C₁-C₆ hydrocarbon-based radicals R₂₂, and hydrogen,
R₁₇, R₁₉, and R₂₁, which may be identical or different, are chosen from linear or branched, saturated or unsaturated C₇-C₂, hydrocarbon-based radicals;
r, n, and p, which may be identical or different, are integers ranging from 2 to 6;
y is an integer ranging from 1 to 10;
x and z, which may be identical or different, are integers ranging from 0 to 10; and
X⁻ is a simple or complex, organic or inorganic anion;
with the proviso that the sum x+y+z ranges from 1 to 15, that when x is 0, then R₁₆ denotes R₂₀, and that when z is 0, then R₁₈ denotes R₂₂.

3. The composition of claim 2, wherein the compound of formula (VIII) is chosen from the salts of diacyloxyethyl-dimethylammonium, of diacyloxyethyl-hydroxyethyl-methyl-ammonium, of monoacyloxyethyl-dihydroxyethyl-methylammonium, of triacyloxyethyl-methylammonium, of monoacyloxyethyl-hydroxyethyl-dimethylammonium, and mixtures thereof.

4. The composition of claim 2, wherein the surfactant of formula (V) is chosen from dipalmitoylethylhydroxyethylmethylammonium salts, behenyltrimethylammonium salts, distearyldimethylammonium salts, cetyltrimethylammonium salts, benzyldimethyl-stearylammonium salts, palmitylamidopropyltrimethylammonium salts, and stearamidopropyldimethyl(myristyl acetate)ammonium salts.

5. The composition of claim 1, wherein the at least one cationic surfactant is chosen from behenyltrimethylammonium chloride, cetyltrimethylammonium chloride, quaternium-83, behenylamidopropyl 2,3-dihydroxypropyl dimethyl ammonium chloride, and palmitylamidopropyltrimethylammonium chloride.

6. The composition of claim 1, wherein the at least one cationic surfactant is present in the composition in an amount ranging from 0.01% to 10% by weight relative to the total weight of the composition.

7. The composition of claim 6, wherein the at least one cationic surfactant is present in an amount ranging from 0.1% to 5% by weight relative to the total weight of the composition.

8. The composition of claim 7, wherein the at least one cationic surfactant is present in an amount ranging from 0.2% to 4% by weight relative to the total weight of the composition.

9. The composition of claim 1, wherein the at least one fatty alcohol is linear or branched, and saturated or unsaturated.

10. The composition of claim 9, wherein the at least one fatty alcohol has the structure R—OH, in which R is chosen from saturated or unsaturated, linear or branched radicals comprising from 8 to 40 carbon atoms.

11. The composition of claim 10, wherein R is chosen from saturated or unsaturated, linear or branched radicals comprising from 8 to 30 carbon atoms.

12. The composition of claim 10, wherein the at least one fatty alcohol is chosen from cetyl alcohol, stearyl alcohol, oleyl alcohol, and mixtures thereof.

13. The composition of claim 1, wherein the at least one fatty alcohol is solid or pasty at room temperature.

14. The composition of claim 1, wherein the at least one fatty alcohol is present in the composition in an amount ranging from 4% to 15% by weight relative to the total weight of the composition.

15. The composition of claim 14, wherein the at least one fatty alcohol is present in an amount ranging from 4.5% to 10% by weight relative to the total weight of the composition.

16. The composition of claim 15, wherein the at least one fatty alcohol is present in an amount ranging from 4.5% to 8% by weight relative to the total weight of the composition.

17. The composition of claim 1, wherein the at least one diol is chosen from 1,6-hexanediol, dipropylene glycol, and mixtures thereof.

18. The composition of claim 1, wherein the at least one diol is present in the composition in an amount ranging from 0.1% to 10% by weight relative to the total weight of the composition.

19. The composition of claim 18, wherein the at least one diol is present in an amount ranging from 0.25% to 5% by weight relative to the total weight of the composition.

20. The composition of claim 19, wherein the at least one diol is present in an amount ranging from 0.25% to 5% by weight relative to the total weight of the composition.

21. The composition of claim 1, wherein the at least one cationic polymer is chosen from:
(1) homopolymers and copolymers derived from at least one entity chosen from acrylic esters, methacrylic esters, acrylic amides, and acrylic esters, and comprising at least one of the units of the following formulae: 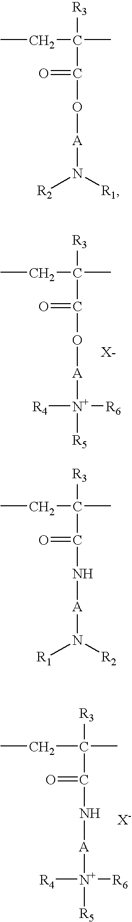
in which:
R₃; which may be identical or different, is chosen from hydrogen and CH₃ radicals;
A, which may be identical or different, is chosen from linear or branched alkyl groups comprising 1 to 6 carbon atoms and hydroxyalkyl groups comprising 1 to 4 carbon atoms;
R₄, R₅, and R₆, which may be identical or different, are chosen from alkyl groups comprising from 1 to 18 carbon atoms and from benzyl radicals;
R₁ and R₂, which may be identical or different, are chosen from hydrogen and alkyl groups comprising from 1 to 6 carbon atoms; and
X⁻ denotes an anion derived from at least one entity chosen from mineral or organic acids and halides;
(2) cationic polysaccharides;
(3) polymers comprising piperazinyl units and divalent alkylene or hydroxyalkylene radicals comprising straight or branched chains, optionally interrupted with at least one atom chosen from oxygen, sulphur, and nitrogen atoms, and/or with at least one ring chosen from aromatic and heterocyclic rings, and the oxidation and/or quaternization products of these polymers;
(4) water-soluble polyamino amides;
(5) polyaminoamide derivatives resulting from the condensation of at least one polyalkylene polyamine with at least one polycarboxylic acid. followed by alkylation with at least one difunctional agent;
(6) polymers obtained by reaction of at least one polyalkylene polyamine comprising two primary amine groups and at least one secondary amine group with a dicarboxylic acid;
(7) cyclopolymers of alkyldiallylamine or of dialkyldiallylammonium;
(8) diquaternary ammonium polymers comprising at least one repeating unit corresponding to formula (II): 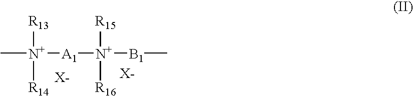
in which:
R₁₃, R₁₄, R₁₅, and R₁₆, which may be identical or different, are chosen from aliphatic radicals comprising from 1 to 20 carbon atoms, alicyclic radicals comprising from 1 to 20 carbon atoms, arylaliphatic radicals comprising from 1 to 20 carbon atoms, and lower hydroxyalkylaliphatic radicals, or alternatively, R₁₃, R₁₄, R₁₅, and R₁₆, together or separately, form, together with the nitrogen atoms to which they are attached, heterocycles optionally comprising a second hetero atom other than nitrogen, or alternatively R₁₃, R₁₄, R₁₅, and R₁₆ are chosen from linear or branched C₁-C₆ alkyl radicals substituted with an entity chosen from nitrile groups, ester groups, acyl groups, amide groups, —CO—O—R₁₇-D groups, and —CO—NH—R₁₇-D groups, wherein R₁₇ is an alkylene group and D is a quaternary ammonium group;
A₁ and B₁, which may be identical or different, are chosen from polymethylene groups comprising from 2 to 20 carbon atoms, which groups may be linear or branched, saturated or unsaturated, and which may comprise, linked to or intercalated in the main chain, at least one entity chosen from aromatic rings, oxygen, sulphur, sulphoxide groups, sulphone groups, disulphide groups, amino groups, alkylamino groups, hydroxyl groups, quaternary ammonium groups, ureido groups, amide groups, and ester groups, and
X⁻ is an anion derived from a mineral or organic acid;
A₁, R₁₃, and R₁₅ can form, together with the two nitrogen atoms to which they are attached, a piperazine ring; and
if A₁ is chosen from linear or branched, saturated or unsaturated alkylene radicals and linear or branched, saturated or unsaturated hydroxyalkylene radicals, then B₁ can be chosen from (CH₂)ₙ—CO-D-OC—(CH₂)ₙ— groups,
in which D is chosen from:
a) glycol residues of formula: —O-Z-O—, wherein Z is chosen from linear or branched hydrocarbon-based radicals and groups corresponding to one of the following formulae:
—(CH₂—CH₂—O)ₓ—CH₂—CH_{2—}
[CH₂—CH(CH₃)—O]_{y}—CH₂—CH(CH₃)—
wherein x and y are integers ranging from 1 to 4, representing a defined and unique degree of polymerization, or alternatively, any number ranging from 1 to 4, representing an average degree of polymerization;
b) bis-secondary diamine residues;
c) bis-primary diamine residues of formula: —NH—Y—NH—, wherein Y is chosen from linear or branched hydrocarbon-based radicals and the divalent radical
—CH₂—CH₂—S—S—CH₂—CH₂—;
d) ureylene groups of formula: —NH—CO—NH—; and
X⁻ is an anion;
(9) polyquaternary ammonium polymers comprising at least one unit of formula (III): 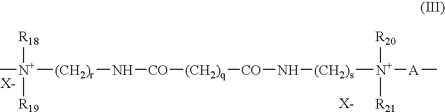
in which:
R₁₈, R₁₉, R₂₀, and R₂₁, which may be identical or different, are chosen from hydrogen, methyl radicals, ethyl radicals, propyl radicals, β-hydroxyethyl radicals, β-hydroxypropyl radicals, and —CH₂CH₂(OCH₂CH₂)ₚOH radicals,
p is equal to an integer ranging from 0 to 6, with the proviso that R₁₈, R₁₉, R₂₀, and R₂₁ are not simultaneously hydrogen,
r and s, which may be identical or different, are integers ranging from 1 to 6,
q is an integer ranging from 0 to 34,
X⁻ is an anion,
A is chosen from dihalide radicals and —CH₂—CH₂—O—CH₂—CH₂—;
(10) quaternary polymers of vinylpyrrolidone and of vinylimidazole;
(11) crosslinked methacryloyloxy(C₁-C₄)alkyltri(C₁-C₄)alkylammonium salt polymers; and
(12) cationic proteins, cationic protein hydrolysates, polyalkyleneimines, polymers comprising vinylpyridine or vinylpyridinium units, condensates of polyamines and of epichlorohydrin, quaternary polyureylenes, and chitin derivatives.

22. The composition of claim 21, wherein in said cationic polymers (1), A is chosen from linear or branched alkyl groups having 2 or 3 carbon atoms.

23. The composition of claim 21, wherein in said cationic polymers (1), R₄, R₅, and R₆, which may be identical or different, are chosen from alkyl groups comprising from 1 to 6 carbon atoms.

24. The composition of claim 21, wherein in said cationic polymers (1), R₁ and R₂, which may be identical or different, are chosen from methyl and ethyl groups.

25. The composition of claim 21, wherein said water-soluble polyamino amides (4) are prepared by polycondensation of at least one acidic compound with at least one polyamine.

26. The composition of claim 1, wherein the at least one cationic polymer is chosen from cationic cyclopolymers, quaternary polymers of vinylpyrrolidone and of vinylimidazole, crosslinked homopolymers and copolymers of methacryloyloxy(C₁-C₄)alkyltri(C₁-C₄)alkylammonium salts, chitosan pyrrolidonecarboxylate, and mixtures thereof.

27. The composition of claim 1, wherein the at least one cationic polymer is present in the composition in an amount ranging from 0.001% to 20% by weight relative to the total weight of the final composition.

28. The composition of claim 27, wherein the at least one cationic polymer is present in an amount ranging from 0.01% to 10% by weight relative to the total weight of the final composition.

29. The composition of claim 1, further comprising at least one silicone.

30. The composition of claim 29, wherein the at least one silicone is an amino silicone.

31. The composition of claim 29, wherein the at least one silicone is chosen from amodimethicones, trimethylsilyl amodimethicones, and silicones comprising quaternary ammonium groups.

32. The composition of claim 29, wherein the at least one silicone is present in the composition in an amount ranging from 0.01% to 6% by weight relative to the total weight of the composition.

33. The composition of claim 32, wherein the at least one silicone is present in an amount ranging from 0.1% to 3% by weight relative to the total weight of the composition.

34. The composition of claim 33, wherein the at least one silicone is present in an amount ranging from 0.4% to 2% by weight relative to the total weight of the composition.

35. The composition of claim 1, wherein the cosmetically acceptable medium is chosen from water, cosmetically acceptable solvents, and water-solvent mixtures.

36. The composition of claim 35, wherein the cosmetically acceptable solvent is chosen from ethanol and isopropanol.

37. The composition of claim 1, further comprising at least one additive chosen from fragrances, UV-screening agents, nonionic surfactants, anionic surfactants, amphoteric surfactants, zwitterionic surfactants, preserving agents, proteins, vitamins, provitamins, nonionic polymers, anionic polymers, cationic polymers, amphoteric polymers, zwitterionic polymers, mineral oils, plant oils, synthetic oils, antidandruff agents, agents for preventing hair loss, dyes, pigments, reducing agents, silicones, plant waxes, ceramides, and polyols other than those of claim 1.

38. The composition of claim 1, wherein the ratio of the weight concentration of the at least one cationic surfactant to the weight concentration of the at least one diol is greater than or equal to 1.

39. A method for the cosmetic treatment or care of the hair and/or scalp comprising applying a cosmetic composition to the hair and/or scalp, wherein the cosmetic composition comprises, in a cosmetically acceptable medium,
at least one cationic surfactant chosen from quaternary ammonium salts,
at least one fatty alcohol,
at least one non-silicone cationic polymer, and
at least one diol comprising six carbon atoms, and
wherein the at least one fatty alcohol is present in an amount greater than or equal to 4% by weight relative to the total weight of the cosmetic composition.

40. The method of claim 39, wherein the composition is applied to the hair and/or scalp after shampooing.

41. A method for the conditioning of keratin fibers comprising applying a cosmetic composition to said fibers, wherein the cosmetic composition comprises, in a cosmetically acceptable medium,
at least one cationic surfactant chosen from quaternary ammonium salts,
at least one fatty alcohol,
at least one non-silicone cationic polymer, and
at least one diol comprising six carbon atoms, and
wherein the at least one fatty alcohol is present in an amount greater than or equal to 4% by weight relative to the total weight of the cosmetic composition.
